(19) United States
(12) Patent Application Publication
(10) Pub. No.: US 2026/0091219 A1
(43) Pub. Date: Apr. 2, 2026

(54) CAGE FOR RIGHT-SIDED CARDIAC PUMP

(71) Applicant: ABIOMED, INC., DANVERS, MA (US)

(72) Inventors: CAM LOUGHLIN, DANVERS, MA (US); CHARLES PERKINS, DANVERS, MA (US); JAIRO LEANDRO MOUX, DANVERS, MA (US); JAMES MELTON, DANVERS, MA (US); NANDISH DESAI, DANVERS, MA (US)

(73) Assignee: ABIOMED, INC., DANVERS, MA (US)

(21) Appl. No.: 19/344,238

(22) Filed: Sep. 29, 2025

Related U.S. Application Data
(60) Provisional application No. 63/701,092, filed on Sep. 30, 2024.

(51) Int. Cl. A61M 60/81; A61M 60/13; A61M 60/216; A61M 60/408

(52) U.S. Cl. CPC A61M 60/81; A61M 60/13; A61M 60/216; A61M 60/408

(57) ABSTRACT

Methods of and systems for performing a medical procedure are herein disclosed. The presently disclosed system generally includes a catheter that is navigated to a target location in a heart of a patient. The catheter extends along a longitudinal axis and includes a catheter body, a pump assembly connected to the catheter body, a cannula connected to the pump assembly and comprising one or more inlet ports, and a cage connected to one or both of the cannula and the pump assembly. When the pump assembly is operated, blood is caused to flow through the cage and through the one or more inlet ports to create a preferential fluid flow through a preferential flow zone of the cage. A biomaterial can thereby be captured at the preferential flow zone.

### CROSS REFERENCE TO RELATED APPLICATIONS

This application claims the benefit of priority under 35 U.S.C. § 119 to prior filed U.S. Provisional Patent Application No. 63/701,092, filed Sep. 30, 2024 (Attorney Docket No.: 266489.000004 (ABD0357USPSP1/D0357.US01)), the entire contents of which is hereby incorporated by reference as if set forth in full herein.

### FIELD

The present disclosure relates generally to medical devices, and in particular to catheters. More specifically, this disclosure relates to a blood pump, in particular an intravascular blood pump for percutaneous insertion into a patient's blood vessel, to support a blood flow in a patient's blood vessel. This disclosure particularly relates to a right ventricular blood pump to support a blood flow from the vena cava through the right ventricle into the pulmonary artery.

### BACKGROUND

Intravascular blood pumps are inserted into a patient's vessel such as the aorta or vena cava and through a cardiac valve by means of a catheter and can also be referred to as catheter pumps. A blood pump typically comprises a pump section with a blood flow inlet and a blood flow outlet. To cause blood flow from the blood flow inlet to the blood flow outlet, typically an impeller or rotor is rotatably supported within the pump casing about an axis of rotation for conveying blood. The blood pump may be driven by a motor included in the blood pump adjacent to the pump section or may alternatively be driven by a motor outside the patient's body, in which case the motor is connected to the impeller by a flexible drive shaft extending through the catheter.

A right ventricular blood pump is inserted through the inferior or superior vena cava through the right ventricle of a patient's heart into the pulmonary artery by means of a catheter. Typically, the blood flow inlet of the blood pump is placed inside the right atrium, vena cava or right ventricle, while the pump section extends through the tricuspid valve, the right ventricle and the pulmonary valve into the pulmonary artery.

Any blood clots or thrombi that occur may be conveyed from the vena cava to the pulmonary artery, which, however does not cause severe harm to the patient because the thrombi only end up in the pulmonary circulation. More importantly, blood clots may clog the blood pump and thus may cause failure of the blood pump, which has to be avoided. Filters are known that can be placed in the vena cava, such as balls of random Nitinol wires. However, handling of such filters adjunctively to the blood pump therapy may be cumbersome, e.g., their insertion and particularly removal would require an additional access point. Any access point carries a risk of bleeding and infection. A correct alignment between blood pump and filter is necessary for performance and needs to be confirmed by means of visualization by echo or x-ray.

### SUMMARY

The disclosed technology includes a catheter that is operable to create a preferential flow zone through a cage to capture biomaterial prior to entry through inlet port(s) of the catheter. By doing so, reduction of performance of the fluid pump assembly of the catheter can be prevented. These and other advantages of the disclosed technology are described herein.

There is provided, in accordance with the disclosed technology, a catheter for insertion into a patient's vasculature. The catheter extends along a longitudinal axis and includes a catheter body, a pump assembly, a cannula, one or more inlet ports, one or more outlet ports, and a cage. The catheter body includes a distal end and a proximal end. The pump assembly disposed at the distal end of the catheter body and includes a distal end portion. The cannula is connected to the distal end portion of the pump assembly and includes a proximal cannula portion and a distal cannula portion. The one or more inlet ports are disposed proximal the proximal cannula portion. The one or more outlet ports are disposed proximal the distal cannula portion. The cage is connected to at least one of the cannula and the pump assembly, disposed over the one or more inlet ports, and includes one or more cage inlet ports. The cage extends along the longitudinal axis and in a circumferential direction about the longitudinal axis and has an asymmetric geometry, relative to the longitudinal axis, in the circumferential direction that is configured to cause a preferential fluid flow to at least one of the one or more cage inlet ports.

There is provided, in accordance with the disclosed technology, a medical system including a catheter and a controller. The catheter extends along a longitudinal axis and includes a catheter body, a pump assembly, a cannula, and a cage. The catheter body includes a distal end and a proximal end. The pump assembly is disposed at the distal end of the catheter body and includes a distal end portion. The cannula is connected to the distal end portion of the pump assembly and includes one or more inlet ports and one or more outlet ports. The cage is connected to at least one of the cannula and the pump assembly so as to surround the one or more inlet ports and includes one or more cage inlet ports. The cage has an asymmetric geometry, relative to the longitudinal axis, in a circumferential direction that is configured to cause a preferential fluid flow to at least one of the one or more cage inlet ports. The controller is connected to the proximal end of the catheter body and is configured to operate the pump assembly.

There is provided, in accordance with the disclosed technology, a method of performing a medical procedure. The method includes navigating a catheter to a target location in a heart of a patient. The catheter extends along a longitudinal axis and includes (i) a catheter body including a distal end and a proximal end, (ii) a pump assembly disposed at the distal end of the catheter body and comprising a distal end portion, (iii) a cannula connected to the distal end portion of the pump assembly and including one or more inlet ports, one or more outlet ports, and a lumen connecting the one or more inlet ports and the one or more outlet ports, and (iv) a cage connected to at least one of the cannula and the pump assembly so as to surround the one or more inlet ports and comprising a first cage inlet port. The method includes operating the pump assembly to pull blood through the first cage inlet port, through the one or more inlet ports, through the lumen of the cannula, and expel the blood through the one or more outlet ports. The method includes capturing a biomaterial at the first cage inlet port.

There is provided, in accordance with the disclosed technology, a method of performing a medical procedure. The method includes navigating a catheter to a target location in a heart of a patient. The catheter extends along a longitudinal axis and includes (i) a catheter body, (ii) a pump assembly connected to the catheter body, (iii) a cannula connected to the pump assembly and comprising one or more inlet ports, and (iv) a cage connected to at least one of the cannula and the pump assembly. The method includes operating the pump assembly to cause blood to flow through the cage and through the one or more inlet ports to create a preferential fluid flow through a preferential flow zone of the cage. The method includes capturing a biomaterial at the preferential flow zone.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements. The figures depict one or more implementations of the inventive devices, by way of example only, not by way of limitation.

FIG. 1 is a schematic pictorial illustration of a catheter inserted into a patient's heart, in accordance with the disclosed technology;

FIG. 2 is a schematic pictorial illustration of a medical system including the catheter of FIG. 1 including a cage, with the cage being shown in cross-section, in accordance with the disclosed technology;

FIG. 3 is a schematic pictorial illustration of a detail view of Detail A in FIG. 2, in accordance with the disclosed technology;

FIG. 4 is a schematic pictorial illustration of a detail view of Detail B in FIG. 3, in accordance with the disclosed technology;

FIG. 5 is a schematic pictorial illustration of an elevation view of the cage, in accordance with the disclosed technology;

FIG. 6A is a schematic pictorial illustration of a section view of the cage, taken along line 6A-6A in FIG. 5, in accordance with the disclosed technology;

FIG. 6B is a schematic pictorial illustration of a section view of the cage, taken along line 6B-6B in FIG. 5, in accordance with the disclosed technology;

FIG. 6C is a schematic pictorial illustration of a section view of an alternative cage configuration, similar to the view of FIG. 5, in accordance with the disclosed technology;

FIG. 6D is a schematic pictorial illustration of a section view of another alternative cage configuration, similar to the view of FIG. 5, in accordance with the disclosed technology;

FIG. 7 is a schematic pictorial illustration of a detail view, similar to the view of FIG. 3, of another alternative cage, in accordance with the disclosed technology;

FIG. 8A is a schematic pictorial illustration, similar to FIG. 3, showing the catheter in use, in accordance with the disclosed technology;

FIG. 8B is a schematic pictorial illustration, similar to FIG. 3, showing the catheter in use, in accordance with the disclosed technology;

FIG. 9 is a flow chart of a method of using a catheter, according to aspects of the present disclosure;

FIG. 10 is a flow chart of another method of using a catheter, according to aspects of the present disclosure;

FIG. 11A is a schematic pictorial illustration of a block diagram depicting an exemplary sensor and cage arrangement, according to aspects of the present disclosure;

FIG. 11B is a schematic pictorial illustration of a block diagram depicting another exemplary sensor and cage arrangement, according to aspects of the present disclosure;

FIG. 11C is a schematic pictorial illustration of a block diagram depicting another exemplary sensor and cage arrangement, according to aspects of the present disclosure; and

FIG. 11D is a schematic pictorial illustration of a block diagram depicting another exemplary sensor and cage arrangement, according to aspects of the present disclosure.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%.

In addition, as used herein, the terms “patient,” “host,” “user,” and “subject” refer to any human or animal subject and are not intended to limit the systems or methods to human use, although use of the subject invention in a human patient represents a preferred embodiment. As well, the term “proximal” indicates a location closer to the operator whereas “distal” indicates a location further away to the operator or physician.

As discussed herein, the terms “tubular” and “tube” are to be construed broadly and are not limited to a structure that is a right cylinder or strictly circumferential in cross-section or of a uniform cross-section throughout its length. For example, the tubular structures are generally illustrated as a substantially right cylindrical structure. However, the tubular structures may have a tapered or curved outer surface without departing from the scope of the present disclosure.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein. Such modifications and variations are intended to be included within the scope of the claims.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein. Such modifications and variations are intended to be included within the scope of the claims.

The disclosed technology relates to a catheter (e.g., an intravascular pump) that is configured to cause a preferential flow of blood caused by a controlled pressure drop at a predetermined location. This enables a predictable capture of biomaterial at the predetermined location and prevention of its entry into the catheter, which can reduce or inhibit its operation. To help the reader better understand the disclosed technology, the catheter will be described, in general, first and then various devices/methods for creating the preferential flow will be described in relation to the intravascular pump. It will be appreciated, however, that the disclosed technology can be applicable to other intravascular pumps having differing designs from that described herein. Furthermore, the disclosed technology can be applicable to other catheters in which entry of biomaterial into a pump is common. Accordingly, the disclosed technology should not be limited to the specific catheter shown in the drawings and described herein.

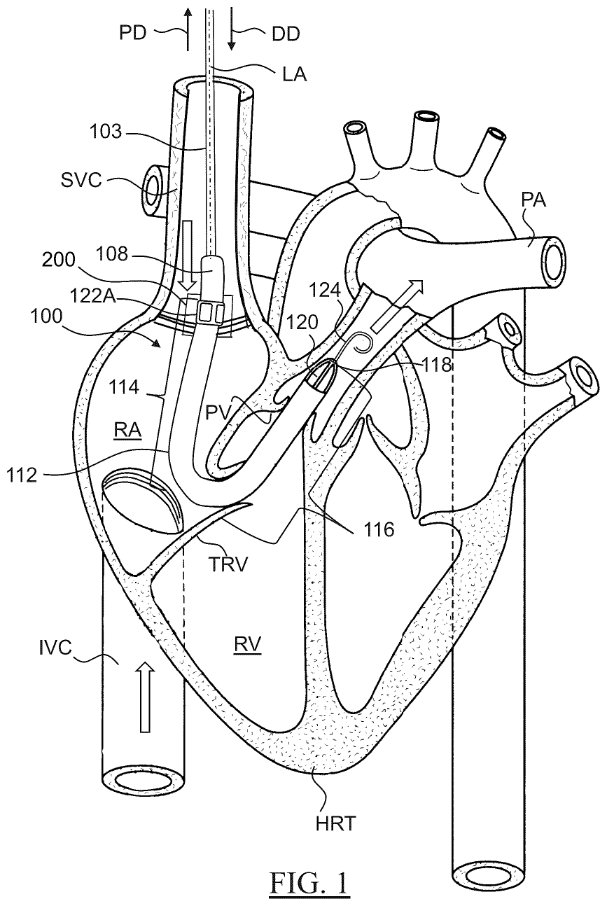

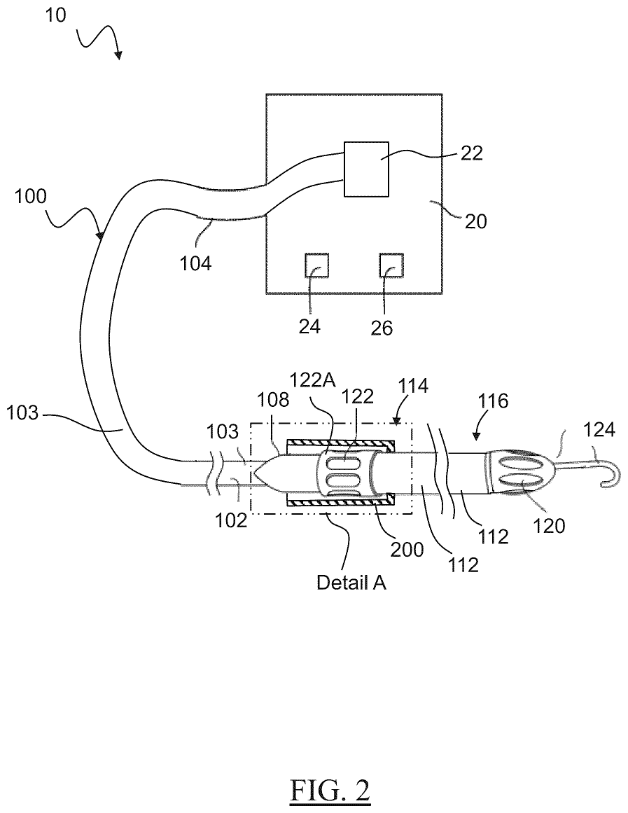

FIG. 1 illustrates a catheter 100 (which can be embodied as, e.g., an intravascular blood pump) inserted into a patient's heart HRT, while FIG. 2 illustrates a medical system 10 that includes the catheter 100 and a controller 20. With reference to FIGS. 1 and 2, the catheter 100 includes a catheter body 103, a pump assembly 108, and a flexible cannula 112. The catheter body 103 extends along a longitudinal axis LA from a proximal end 104 to a distal end 102 (labelled in FIG. 2). The pump assembly 108 is coupled to the distal end 102 of the catheter body 103 and the controller 20 is coupled to the proximal end 104 of the catheter body 103. The cannula 112 is coupled to a distal end portion 110 of the pump assembly 108. The cannula 112 has a proximal cannula portion 114 and a distal cannula portion 116 and defines a lumen 112A. The proximal cannula portion 114 and distal cannula portion 116 may be approximately parallel to the longitudinal axis LA of the catheter body 103 and may be approximately straight.

The catheter 100 further includes one or more inlet ports 122 disposed on or near the proximal cannula portion 114 and one or more outlet ports 120 disposed on or near the distal cannula portion 116 at a distal end of the catheter 100. In the present example, the blood flow inlet port(s) 122 and the blood flow outlet port(s) 120 are formed as circumferential openings in respective cages 122A, 120A. It will be appreciated that other shapes, sizes or positions may be suitable for the inlet ports 122 and outlet ports 120, possibly depending on the application. In the present example, the cages 120A, 122A are separate from the cannula 112; however, it will be appreciated that, in other examples, the ports 120, 122 can be defined integrally with the cannula 112.

The shape, size, and material of the cannula 112 is optimized for insertion of the catheter 100 into the right heart of the patient such that the cannula 112 traverses the superior vena cava SVC, right atrium RA, tricuspid valve TRV, right ventricle RV, and pulmonary valve PV of the heart HRT. This allows the inlet ports 122 to be positioned in the superior vena cava SVC, the inferior vena cava, or the right atrium RA and the outlet ports 120 to be positioned in the pulmonary artery PA.

In some implementations, the length of the cannula 112 is 22 cm. In certain implementations, the length of the cannula is in the range of 17-25 cm or any other suitable length. The cannula 112 is constructed to allow fluid to flow into the inlet ports 122, through the cannula 112, and out the outlet ports 120. The fluid may be propelled through the cannula 112 by an impeller 109 of the pump assembly 108 (discussed in greater detail below) located in the pump assembly 108. In some examples, the cannula 112 may be configured to be relatively stiff to increase the stability of the cannula 112 once in place in the right heart.

The cannula 112 is also sized for passage through a femoral artery and other vasculature of a patient. In some implementations, the cannula 112 has a cannula diameter of about 22 Fr. In certain implementations, the cannula 112 has a cannula diameter of 7 Fr, 8 Fr, 9 Fr, 10 Fr, 11 Fr, 12 Fr, 18 Fr, 20 Fr, 21 Fr, 22 Fr, 23 Fr, 24 Fr, or any other suitable diameter. The cannula diameter may be approximately constant along the length of the cannula 112.

In certain implementations, the cage 120A defining the outlet ports 120 of the catheter 100 may narrow toward a distal end of the outlet ports 120, which can further facilitate passage through the heart valves. In certain implementations, a flexible extension 124 can be connected to the outlet ports 120 to prevent traumatic contact of the distal end of the catheter 120 with interior walls of the heart following insertion.

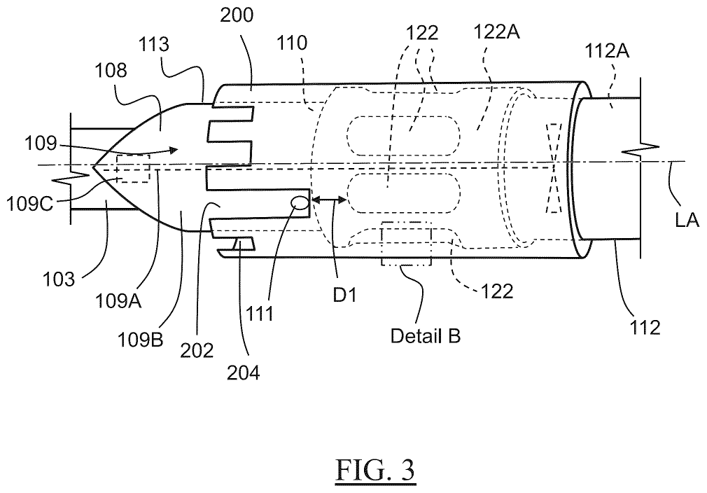

As seen in phantom lines in FIG. 3, the pump assembly 108 can contain a rotor 109A which may be driven by an implantable or external drive unit. In some examples, the pump assembly includes an impeller 109 having (i) the rotor 109A, with the rotor 109A being connected to the distal end 102 of the catheter body 103, (ii) one or more impeller blades 109B connected to and rotatable by the rotor 109A, and (iii) a motor 109C connected to the rotor 109A that causes rotation thereof. The pump assembly 108 includes a housing 113 which may be comprised of a different material than the cannula 112 or catheter 103. The rotor 109A of the pump assembly 108 may be located in the housing 113 and attached to a drive shaft (not shown). Although an example with an implantable motor 109C is shown in FIG. 3, in some implementations the unit driving the drive shaft is located external to the patient's body and the drive shaft extends through the catheter body 103. In some implementations, a motor 109C driving the drive shaft is enclosed within the pump assembly 108 (as shown). However, any suitable pump and/or drive known in the art may be used.

The pump assembly 108 is configured to provide a fluid flow into the cannula 112 at the inlet ports 122, through the cannula 112, and out the outlet ports 120. The pump assembly 108 may be configured to provide a flow rate of 4 liters per minute (1 pm) or more within the right heart of a patient. In some implementations, the pump assembly 108 provides a flow rate of 3 lpm, 3.5 lpm, 4 lpm, 4.5 lpm 5 lpm, 6 lpm or any other suitable flow rate. In some implementations, the flow rate is chosen based on the needs of the patient.

The controller 20 is functional to control operation of the pump assembly 108. The controller includes a fluid reservoir 22 that is configured to contain a purge fluid for purging the motor 109C, which prevents buildup on the rotor 109A and/or impeller 109B and also cools the motor 109C. In some examples, the purge fluid includes heparin, sodium bicarbonate, or any other appropriate fluid and/or combinations thereof in various proportions. The controller 20 is configured to deliver the purge fluid through the pump assembly and/or the motor 109C. The controller 20 can include a fluid supply control 24 for controlling supply of the purge fluid to the pump assembly and a flow rate control 26 for controlling operation of the pump assembly 108. Further exemplary details of the purging of the pump assembly 108 can be found in U.S. Patent Publication No. 2021/0339003 A1, which is incorporated herein by reference. In operation, the controller 20 controls the pump assembly 108 to pull a fluid (e.g., blood) through the inlet ports 122, through the lumen 112A of the cannula 112 and expel the fluid through the outlet ports 120.

Further to the above and as seen in FIG. 1, in use, the catheter 100 is inserted into the right ventricle RV of the patient's heart HRT in a distal direction DD via the superior vena cava SVC and right atrium RA (as shown). In a different approach, the catheter may be inserted through the inferior vena cava IVC. During its operation, cannula 112 placed through the tricuspid valve TRV and the pulmonary valve PV. The pump assembly 108, via the impeller 109 (FIG. 3), causes the blood to flow into the inlet ports 122 towards and out of the outlet ports 120 (see the arrows in FIG. 1). To remove and/or adjust a position of the catheter 100, it can be moved in a proximal direction PD.

In some instances, while the pump assembly 108 is being operated, biomaterial (e.g., thrombus) can undesirably enter through the inlet ports 122, which can become trapped in and/or occlude the motor 109C and/or on the impeller blades 109B. This can cause a significant decrease in the flow rate of the pump assembly 108. It is desirable, therefore, to provide for a controlled pressure drop at a predefined location that enables the capture of the biomaterial at the predefined location, prior to entry through the inlet ports 122. In examples detailed in the present disclosure, the predefined location is provided in a cage that surrounds the inlet ports 122. Moreover, in some examples, detection techniques (e.g., the use of optical signals) can be employed to determine when the biomaterial is captured at the predefined location.

FIG. 3 is a schematic pictorial illustration of an inlet portion of the catheter 100 that includes a cage 200, with portions of the catheter 100 hidden by the cage 200 and/or the pump assembly housing 113 being shown in phantom lines. More particularly, FIG. 3 is a detail view of Detail A in FIG. 2, with the exception of the cage 200 being shown in full rather than in cross-section (as in FIG. 2). With particular reference now being made to FIGS. 2 and 3, the cage 200 extends along the longitudinal axis LA and is connected to at least one of the cannula 112 and the pump assembly 108. In some examples, the cage 200 is bonded and/or adhered onto the cannula and/or the pump assembly 108. In some examples, the cage 200 includes a biocompatible metal (e.g., nickel titanium or stainless steel) or a biocompatible plastic material (e.g., flexible biocompatible plastics, such as, but not limited to, poly (methyl methacrylate) (PMMA) or thermoplastic polyurethane (TPU)). As seen best in FIG. 3, the cage 200 is dimensioned and positioned to be disposed substantially and/or entirely over the one or more inlet ports 122 along the longitudinal axis LA. Put another way, the cage 200 extends in a circumferential direction CD about the longitudinal axis to substantially surround the inlet ports 122.

The cage 200 includes one or more cage inlet ports 202, 204 disposed circumferentially around the cage 200. While the presently illustrated examples include multiple cage inlet ports, in some examples, a singe first cage inlet port 202 can be employed without departing from the spirit and scope of the present disclosure.

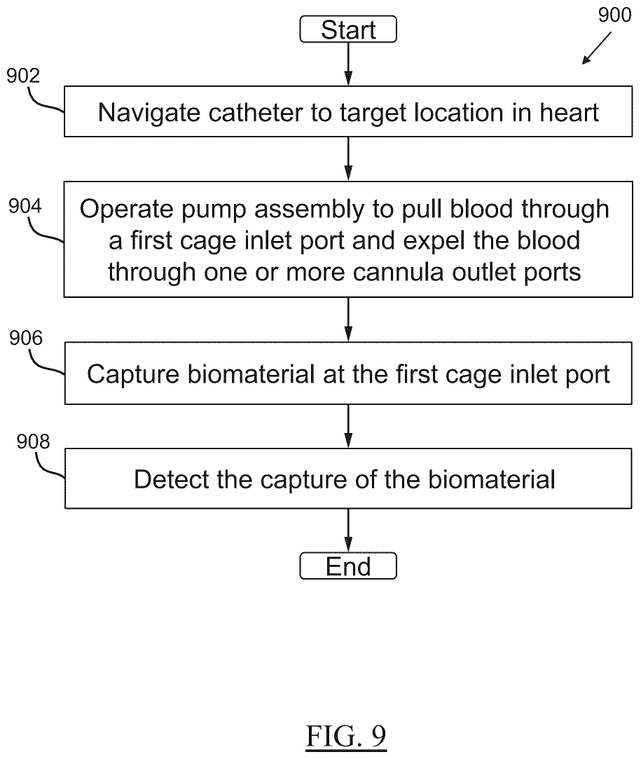

In some examples, and as discussed in greater detail below, the cage 200 has an asymmetric geometry relative to the longitudinal axis LA in the circumferential direction CD. This asymmetric geometry is designed such that a preferential fluid flow PFF (FIG. 8A) is caused to at least one of the cage inlet ports 202 (referred to herein as the first cage inlet port 202) while a fluid flow FF, different from the preferential fluid flow PFF, is caused at the other, second cage inlet ports 204 (and, in general, a remainder of the cage 200 in the circumferential direction CD thereof) when the pump assembly 108 is operated.

As will be appreciated by those skilled in the art, a similar effect can also be achieved by, e.g., configuring the inlet ports 122 with asymmetric geometry relative to the longitudinal axis LA in the circumferential direction CD which also causes a preferential fluid flow PFF to the first cage inlet port 202. It is noted that these alternative configurations can be similarly designed as the following examples 200 of the cage and are fully within the spirit and scope of the presently disclosed technology.

By creating the preferential fluid flow PFF to a predefined, preferential first cage inlet port 202, a biomaterial (e.g., thrombus THR, it is noted that biomaterial hereafter is referred to as biomaterial THR, but can include biomaterial other than thrombus) is able to be predictably captured thereat. More specifically, the preferential fluid PFF can be comprised of a higher pressure drop/gradient from the from the first cage inlet port 202 to a closest inlet port 122A of the inlet ports 122 than any other pressure drop/gradient from the other second cage inlet ports 204 to their respective closest inlet port 122. As will be appreciated by those skilled in the art, the preferential fluid flow PFF can be caused in a plurality of ways without departing from the spirit and scope of the present disclosure. The following examples are non-limiting and merely intended to describe some ways in which the disclosed technology can be implemented in a catheter 100 that includes a pump 108.

With continued reference to FIG. 3, the cage inlet ports 202, 204 are disposed closer to the pump assembly 108 than the inlet ports 122 along the longitudinal axis LA. When inserted into the heart HRT, the cage inlet ports 202, 204 are upstream of the inlet ports 122, i.e., proximally with respect of the inlet ports 122, to require blood and other biomaterial THR to pass through the cage inlet ports 202, 204 prior to passing through the inlet ports 122.

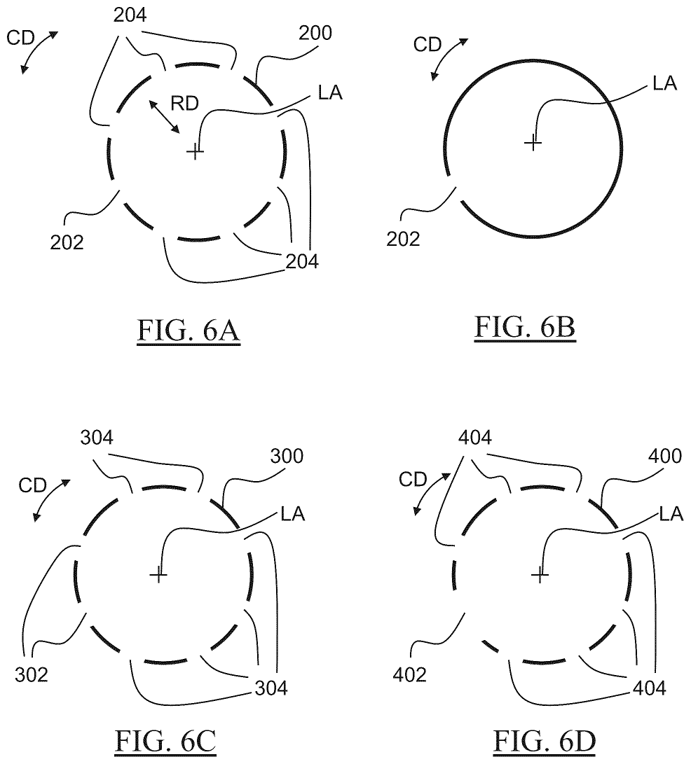

As seen best when viewing FIG. 5 in conjunction with FIG. 3, the first cage inlet port 202 extends from a distal end 202A to a proximal end 202B. Similarly, the other second cage inlet ports 204 extends from respective distal ends 204A to proximal ends 204B. In some examples, such as the one depicted in FIGS. 2-5, the cage inlet ports 202, 204 are formed in a proximal end of the cage 200 such that the proximal end 202B is open and the distal end is closed. It will be appreciated, however, and as discussed below, that the cage inlet ports 202, 204 may have other configurations without departing from the spirit and scope of the disclosed technology. In these and similar examples, the distal ends 202A, 202B of each cage inlet port 202, 204 are spaced from the inlet ports 122 at least a predetermined distance D1 along the longitudinal axis LA. In this example, the preferential cage inlet port 202 is spaced the predetermined distance D1 from the inlet ports 122. However, in other examples, one or more of the cage inlet ports 202, 204 can partially or entirely overlap with the inlet ports 122 depending on the design and/or the pressure differential(s) needed for optimal capture of the biomaterial THR.

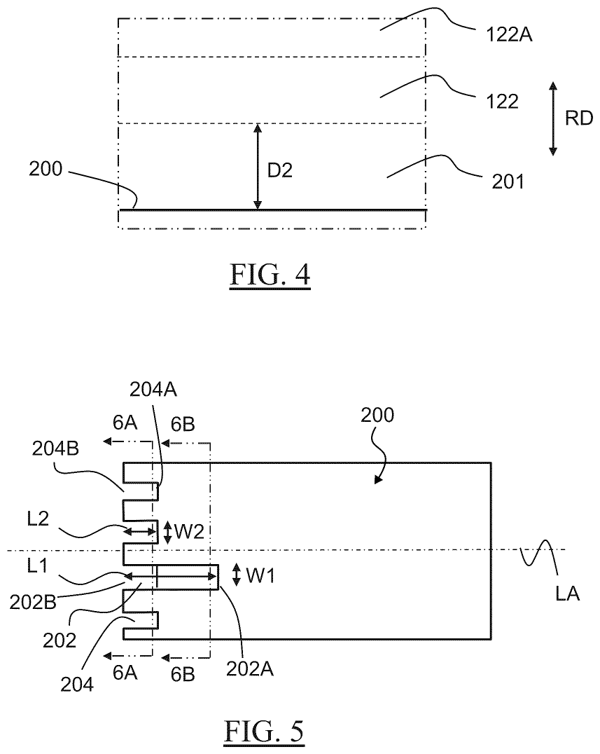

Fluidically connecting the inlet ports 122 and the cage inlet ports 202, 204 is a conduit 201. As best seen in FIG. 4, the conduit 201 is defined between an inner wall of the cage 200 and the inlet cage 122A so as to form an annular-shape. The conduit spaces the at least a portion of the cage from each inlet port by at least a predetermined distance D2 in a radial direction RD that is perpendicular to the longitudinal axis LA. In some examples, the predetermined distance D2 can fall within the range of approximately a few microns (e.g., 5-10 microns) to approximately 3 millimeters. In other examples, D2 can be as large as approximately 1 centimeter. As those skilled in the art will appreciate, the predetermined distance D2 can be adjusted to design for different flow rate and/or pressure requirements, as needed.

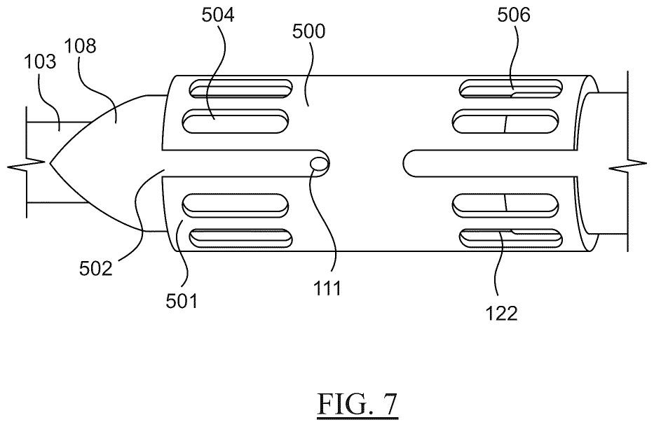

Returning to FIG. 5 and making reference to FIG. 6B, the first cage inlet port 202 has a length L1 and a width W1, while the second cage inlet ports 204 have a length L2 and a width W2. In some examples, the first cage inlet port 202 has a length L1 that is longer than a length L2 of all the second cage inlet ports 204. See, for example, FIGS. 6A 6B, which show all of the cage inlet ports 202, 204 to where section 6A-6A is cut in FIG. 5, whereas only the first cage inlet port 202 extends to where section 6B-6B is cut in FIG. 5. In this example, the longer length L1, in conjunction with the distal end 202A of the first cage inlet port 202 being closer to the inlet ports 122 than the distal ends 204A of the second cage inlet ports 204, results in the preferential fluid flow PFF. Each cage inlet port 202, 204 also has a width W1, W2, respectively.

As seen particularly in FIG. 3, the catheter 100 further includes a sensor 111 approximately aligned with the distal end 202A of the first cage inlet port 202 along the longitudinal axis LA. This sensor 111 can be used to detect the presence of the capture of biomaterial THR. As will appreciated by those skilled in the art, the sensor 111 can be an optical sensor, a pressure sensor, or any other appropriate type of sensor. As will also be appreciated, the sensor 111 may be positioned in any suitable location on the catheter 100.

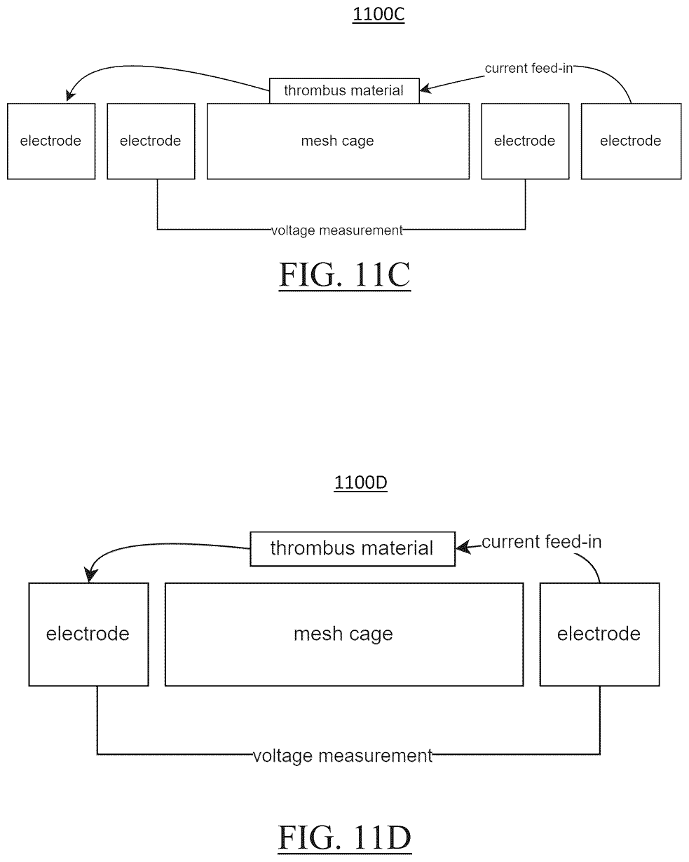

In some examples, the cage 200 may be made of a conductive material and the sensor 111 may be an electrode. See, for example, the exemplary schematic arrangement 1100A depicted in FIG. 11A. The sensor 111 may be located at a distance from the cage 200. In such examples, the cage 200 and the sensor 111 may form an electrode pair. The electrode may generate an electric field. In such examples, the electrode pair may sense contact with tissues when the electric field is disrupted. The tissue may be biomaterial. For example, the electrode pair may be able to detect the presence of the capture of biomaterial THR. The electrode pair may be able to detect a change in measured impedance. As will be appreciated, any suitable number of electrode pairs may be used. See, for example, the exemplary schematic arrangement 1100B depicted in FIG. 11B that illustrates two electrode pairs. The electrode pair may include a plurality of sensors 111. In such examples, the electrode pair may include two electrodes, the cage 200 and an electrode, or some combination of the two. The one or more electrodes may be ring-shaped. The one or more electrodes may be configured to cover at least a portion of the circumference of the cage 200 and/or first cage inlet port 202. As discussed herein, the cage 200 may be made of a mesh material.

In other examples, the cage 200 may not be made of a conductive material. In such examples, the catheter 100 may include one or more electrode pairs. See, for example, the exemplary schematic arrangement 1100C depicted in FIG. 11C that illustrates a four-electrode configuration and the exemplary schematic arrangement 1100D depicted in FIG. 11D that illustrates a two-electrode configuration. As mentioned herein, the one or more electrodes may be ring-shaped. The one or more electrodes may be configured to cover at least a portion of the circumference of the cage 200 and/or the first cage inlet port 202. The one or more electrodes may all have the same geometry (e.g., all electrodes are ring-shaped). However, as will be appreciated, the one or more electrodes may have different geometries. The electrodes may be positioned at any suitable location along the length of the cage 200. In other examples, the electrodes may be positioned at any suitable location along the catheter 100.

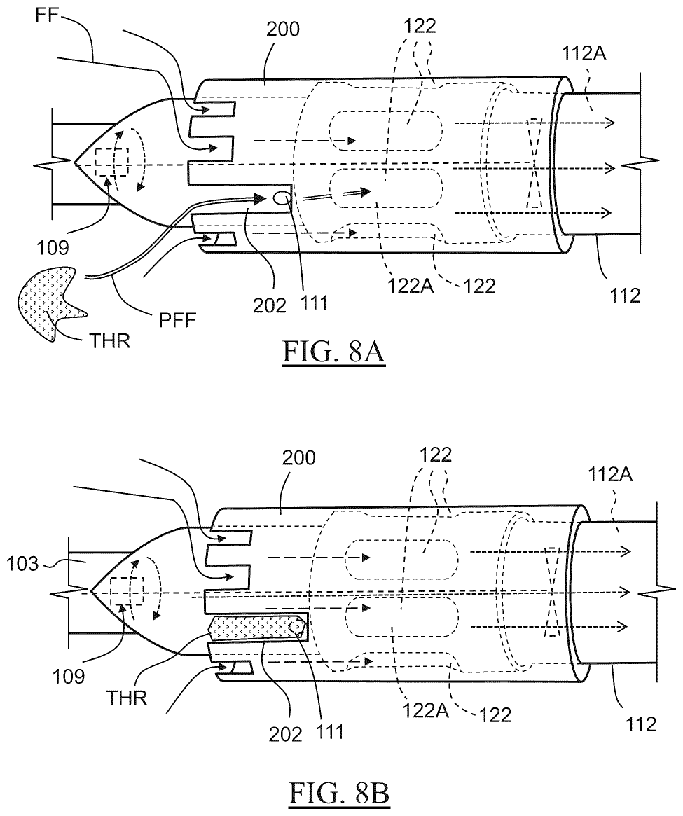

As mentioned above, the cage 200 (and, in general, the presently described system) are not necessarily limited to the configurations described in the foregoing paragraphs. Further to the above, FIGS. 6C-7 depict other exemplary configurations of the cage 200 in accordance with the presently disclosed technology.

Whereas the cage 200 depicted in, e.g., FIGS. 3-6B, includes one first cage inlet port 202, FIG. 6C depicts an alternative configuration 300 of the cage 200 where there are two first cage inlet ports 302 disposed adjacent one another along the circumferential direction CD. This configuration 300 provides various benefits, such as providing for a “backup” first cage inlet port 302 in the event that one becomes blocked/occluded.

FIG. 6D depicts another alternative configuration 400 of the cage 200. In this configuration 400, the first cage inlet port 402 has a width W1 wider than a width W2 of each second cage inlet port 404.

Moreover, FIG. 7 depicts yet another alternative configuration 500 of the cage. With this configuration 500, the first cage inlet port 502 is defined in and through the proximal end 501 thereof, while the second cage inlet ports 504 are spaced from the proximal end 501 such that they have a closed proximal end and a closed distal end along the longitudinal axis LA. This configuration 500 also includes a second plurality of cage inlet ports 506 that are spaced from the first and second cage inlet ports 502, 504 along the longitudinal axis LA. These ports 506 function as a secondary protection against biomaterial THR from being ingested into the inlet ports 122 through their geometry and by virtue of their allowing additional flow to prevent the generation of excessive pressure at a single inlet port 502, 504.

FIGS. 8A-8B illustrates an exemplary use case of the cage of the present disclosure. While the cage 200 of FIG. 3 is depicted, it will be appreciated by those skilled in the art that any of the foregoing cages 200, 300, 400, 500 and/or other alternative configurations (e.g., asymmetric design of the inlet ports 122) can analogously employed without departing from the spirit and scope of the present disclosure.

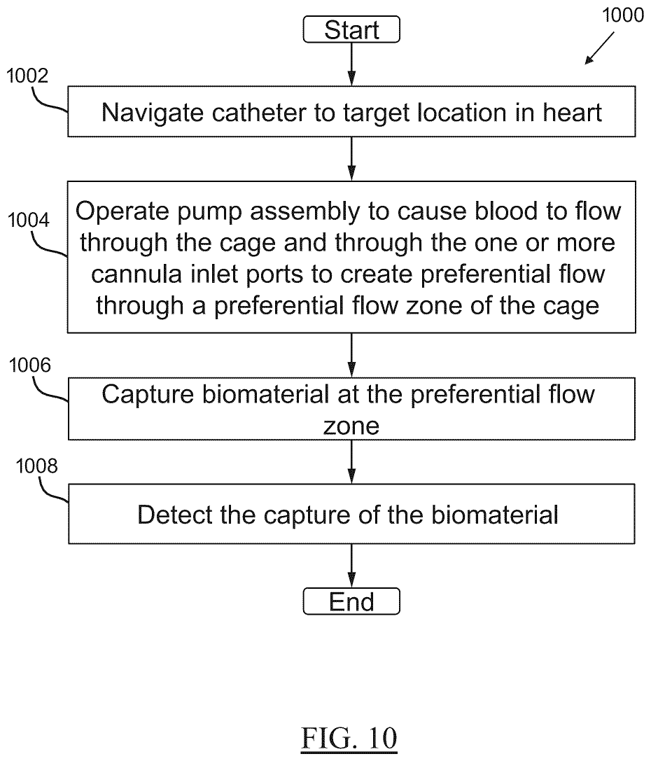

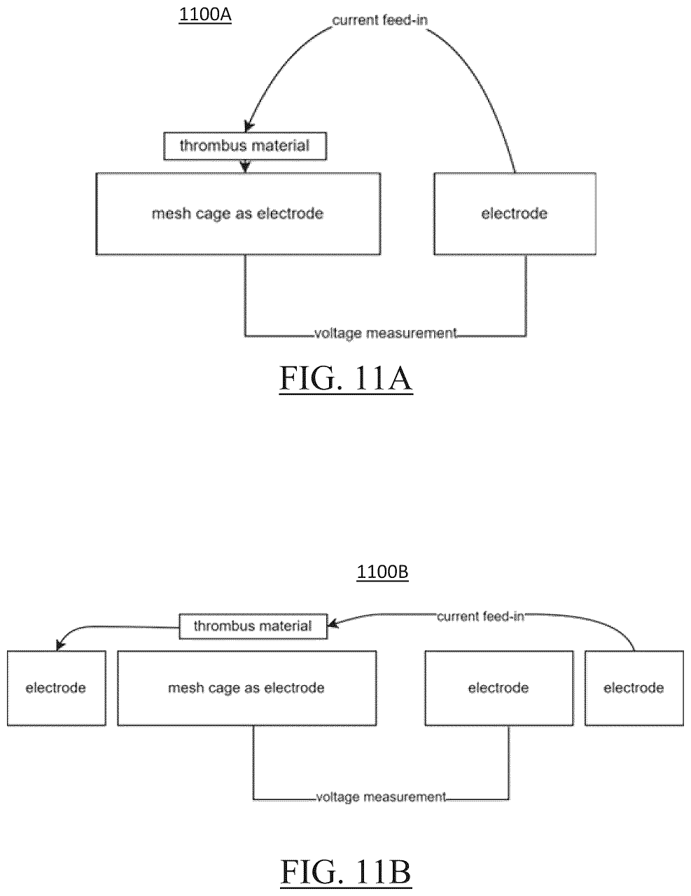

FIGS. 9-10 illustrate example methods 900, 1000 of performing a medical procedure in accordance with the present disclosure.

Making reference to FIG. 9, the method 900 can include navigating 902 the catheter 100 to a target location (e.g., as shown in FIG. 1, where the target location is the pulmonary artery PA) in a heart HRT of a patient. In some examples, navigating 902 can include navigating through the superior vena cava SVC, through the right atrium RA, and through the pulmonary vein PV. As mentioned above, the catheter 100 can take any of the previously described forms, such as the catheter 100 depicted in FIG. 2. The pump assembly is operated 904 (see the rotational arrows in FIGS. 8A and 8B showing the rotor 109A spinning) to pull blood such that it passes (i) through the cage inlet ports 202, 204, including the first cage inlet port 202 (see the solid line arrows in FIGS. 8A and 8B showing blood being directed to and through the cage inlet ports 202, 204), (ii) through the inlet ports 122 (see the long-dash arrows in FIGS. 8A and 8B showing blood being directed to and through the inlet ports 122), (iii) through the lumen 112A of the cannula 112 (see the short-dash arrows in FIGS. 8A and 8B showing blood being directed through the cannula 112A), and (iv) is expelled through the outlet ports 120 (FIG. 2).

As denoted by the double line arrows in FIG. 8A (compared with the single line arrows denoting regular fluid flow FF), a preferential fluid flow PFF is created in a particular zone (i.e., the region where the double line arrows are shown) of the system 10 when the catheter 100 is in use, by virtue of one of the configurations described herein and/or other configurations that would be apparent to those skilled in the art in view of the present disclosure. By providing the inlet in a more proximal position relative to the inlet, the biomaterial THR (e.g., thrombus) has a more tortuous path which increases the likelihood of its capture. Therefore, as seen in FIG. 8B, the method 900 further can include capturing 906 the biomaterial THR at the first cage inlet port 202. Following/concurrently with capture, in some examples, the method 900 can further include detecting 908 the capture of the biomaterial 908 via the sensor 111.

Making reference to FIG. 10, a similar method 1000 to that of method 900 is illustrated. The method 1000 can include navigating 1002 the catheter 100 to a target location (e.g., as shown in FIG. 1, where the target location is the pulmonary artery PA) in a heart HRT of a patient. In some examples, navigating 1002 can include navigating through the superior vena cava SVC, through the right atrium RA, and through the pulmonary vein PV. The pump assembly is operated 1004 (see the rotational arrows in FIGS. 8A and 8B showing the rotor 109A spinning) to pull blood through the cage (e.g., cage 200) to create a preferential fluid flow PFF through a preferential fluid flow zone of the cage (e.g., see the region defined in and around the cage where the double line arrow is, such as a region defined by the first cage inlet port 202). The method 1000 further can include capturing 1006 the biomaterial THR at the preferential flow zone. In some examples, and as discussed above, the preferential fluid flow PFF can be caused by a larger pressure drop in the preferential flow zone from a proximal end of the preferential flow zone (e.g., left side of FIG. 8A) to a closest one 122A of the inlet ports 122 than a pressure drop from any other remaining location along a proximal end of the cage (e.g., cage 200) to its respective closest inlet port 122.

The following clauses list non-limiting examples in accordance with the disclosed technology:
Clause 1. A catheter for insertion into a patient's vasculature, the catheter extending along a longitudinal axis and comprising: a catheter body comprising a distal end and a proximal end; a pump assembly disposed at the distal end of the catheter body and comprising a distal end portion; a cannula connected to the distal end portion of the pump assembly and comprising a proximal cannula portion and a distal cannula portion; one or more inlet ports disposed proximal the proximal cannula portion; one or more outlet ports disposed proximal the distal cannula portion; and a cage connected to at least one of the cannula and the pump assembly, disposed over the one or more inlet ports, and comprising one or more cage inlet ports, the cage (i) extending along the longitudinal axis and in a circumferential direction about the longitudinal axis and (ii) having an asymmetric geometry, relative to the longitudinal axis, in the circumferential direction that is configured to cause a preferential fluid flow to at least one of the one or more cage inlet ports.
Clause 2. The catheter of clause 1, wherein the preferential fluid flow is configured to capture a biomaterial at the at least one of the one or more cage inlet ports.
Clause 3. The catheter of clause 2, wherein the at least one of the one or more cage inlets is disposed at a first location along a circumference of a proximal end of the cage, and the remaining locations along the circumference of the proximal end of cage are configured to cause a fluid flow, different from the preferential fluid flow, to the remaining locations.
Clause 4. The catheter of clause 3, wherein the preferential fluid flow comprises a higher pressure drop from the at least one of the one or more cage inlet ports to its closest inlet port than a pressure drop from any other remaining location along the circumference of the proximal end of the cage to its respective closest inlet port.
Clause 5. The catheter of any one of clauses 1-4, wherein the one or more cage inlet ports are disposed closer to the pump assembly than the one or more inlet ports along the longitudinal axis.
Clause 6. The catheter of any one of clauses 1-5, wherein each cage inlet port comprises a distal end spaced at least a predetermined distance from the one or more inlet ports along the longitudinal axis.
Clause 7. The catheter of any one of clauses 1-6, wherein at least a portion of the cage is spaced from each inlet port so as to define a conduit that fluidically connects the one or more cage inlet ports with the one or more inlet ports.
Clause 8. The catheter of clause 7, wherein the conduit spaces the at least a portion of the cage from each inlet port by at least a predetermined distance in a radial direction that is perpendicular to the longitudinal axis, the predetermined distance being in a range of approximately 5 microns to 3 millimeters.
Clause 9. The catheter of any one of clauses 7-8, wherein the conduit is approximately annular-shaped.
Clause 10. The catheter of any one of clauses 1-9, wherein the one or more inlet ports are formed in an inlet cage.
Clause 11. The catheter of any one of clauses 1-10, wherein the one of the one or more cage inlet ports comprising a first cage inlet port, and wherein the one or more cage inlet ports further comprise one or more second cage inlet ports disposed circumferentially about the cage relative to the first cage inlet port.
Clause 12. The catheter of clause 11, wherein the one or more second cage inlet ports comprise a plurality of second cage inlet ports.
Clause 13. The catheter of any one of clauses 11-12, further comprising an additional first cage inlet port disposed adjacent the first cage inlet port along the circumferential direction.
Clause 14. The catheter of any one of clauses 11-13, wherein the first cage inlet port comprises an open proximal end and a closed distal end along the longitudinal axis.
Clause 15. The catheter of any one of clauses 11-14, wherein the one or more second cage inlet ports comprise an open proximal end and a closed distal end along the longitudinal axis.
Clause 16. The catheter of any one of clauses 11-14, wherein the one or more second cage inlet ports comprise a closed proximal end and a closed proximal end along the longitudinal axis.
Clause 17. The catheter of any one of clauses 11-16, wherein the cage further comprises a second plurality of cage inlet ports spaced from the plurality of cage inlet ports along the longitudinal axis.
Clause 18. The catheter of any one of clauses 11-17, wherein the first cage inlet port comprises a length longer than a length of each second cage inlet port.
Clause 19. The catheter of any one of clauses 11-18, wherein a distal end of the first cage inlet port is closer to the one or more inlet ports than a distal end of each second cage inlet ports.
Clause 20. The catheter of any one of clauses 1-10, wherein the one of the one or more cage inlet ports comprises solely a first cage inlet port.
Clause 21. The catheter of any one of clauses 11-20, further comprising a sensor approximately aligned with a distal end of the first cage inlet port along the longitudinal axis.
Clause 22. The catheter of clause 21, wherein the sensor comprises one of an optical sensor and a pressure sensor.
Clause 23. The catheter of any one of clauses 1-23, wherein the pump assembly is configured to pull a fluid through the one or more inlet ports, through a lumen of the cannula, and expel the fluid through the one or more outlet ports.
Clause 24. The catheter of any one of clauses 1-23, wherein the pump assembly comprises: a rotor connected to the distal end of the catheter body; and one or more impeller blades connected to the rotor.
Clause 25. The catheter of clause 24, wherein the pump assembly comprises a motor connected to the rotor.
Clause 26. The catheter of any one of clauses 1-25, the cage comprising a biocompatible metal material.
Clause 27. The catheter of clause 26, the biocompatible metal material comprising nickel titanium or stainless steel.
Clause 28. A medical system comprising: a catheter extending along a longitudinal axis and comprising: a catheter body comprising a distal end and a proximal end; a pump assembly disposed at the distal end of the catheter body and comprising a distal end portion; a cannula connected to the distal end portion of the pump assembly and comprising one or more inlet ports and one or more outlet ports; and a cage connected to at least one of the cannula and the pump assembly so as to surround the one or more inlet ports and comprising one or more cage inlet ports, the cage having an asymmetric geometry, relative to the longitudinal axis, in a circumferential direction that is configured to cause a preferential fluid flow to at least one of the one or more cage inlet ports; and a controller connected to the proximal end of the catheter body and configured to operate the pump assembly.
Clause 29. The medical system of clause 28, wherein the controller comprises a fluid reservoir that is configured to contain a purge fluid, the controller being configured to deliver the purge fluid through the pump assembly.
Clause 30. The medical system of clause 29, wherein the controller comprises a fluid supply control for controlling supply of the purge fluid to the pump assembly and a flow rate control for controlling operation of the pump assembly.
Clause 31. The medical system of any one of clauses 28-30, wherein the controller is configured to operate the pump assembly to pull a fluid through the one or more inlet ports, through a lumen of the cannula, and expel the fluid through the outlet port.
Clause 32. A method of performing a medical procedure comprising: navigating a catheter to a target location in a heart of a patient, the catheter extending along a longitudinal axis and comprising (i) a catheter body comprising a distal end and a proximal end, (ii) a pump assembly disposed at the distal end of the catheter body and comprising a distal end portion, (iii) a cannula connected to the distal end portion of the pump assembly and comprising one or more inlet ports, one or more outlet ports, and a lumen connecting the one or more inlet ports and the one or more outlet ports, and (iv) a cage connected to at least one of the cannula and the pump assembly so as to surround the one or more inlet ports and comprising a first cage inlet port; operating the pump assembly to pull blood through the first cage inlet port, through the one or more inlet ports, through the lumen of the cannula, and expel the blood through the one or more outlet ports; capturing a biomaterial at the first cage inlet port.
Clause 33. The method of clause 32, further comprising: detecting the capture of the biomaterial.
Clause 34. The method of any one of clauses 32-33, wherein the target location comprises the pulmonary artery of the heart.
Clause 35. The method of any one of clauses 32-34, wherein navigating the catheter to the target location comprises navigating through the superior vena cava, through the right atrium, and through the pulmonary vein.
Clause 36. The method of any one of clauses 32-35, wherein the biomaterial comprises a thrombus.
Clause 37. The method of any one of clauses 32-36, wherein the cage comprises an asymmetric geometry, relative to the longitudinal axis, in a circumferential direction of the cage.
Clause 38. The method of any one of clauses 32-37, wherein the cage comprises a plurality of second cage inlet ports, and the first cage inlet port is disposed closer to its closest inlet port than each of the second cage inlet ports and its respective closest inlet ports, creating a preferential fluid flow to the first cage inlet port.
Clause 39. A method of performing a medical procedure comprising: navigating a catheter to a target location in a heart of a patient, the catheter extending along a longitudinal axis and comprising (i) a catheter body, (ii) a pump assembly connected to the catheter body, (iii) a cannula connected to the pump assembly and comprising one or more inlet ports, and (iv) a cage connected to at least one of the cannula and the pump assembly; operating the pump assembly to cause blood to flow through the cage and through the one or more inlet ports to create a preferential fluid flow through a preferential flow zone of the cage; capturing a biomaterial at the preferential flow zone.
Clause 40. The method of clause 39, wherein the preferential fluid flow is caused by a larger pressure drop in the preferential flow zone from a proximal end of the preferential flow zone to a closest one of the one or more inlet ports than a pressure drop from any other remaining location along a proximal end of the cage to its respective closest inlet port.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes. Therefore, to the extent there are variations of the disclosed technology, which are within the spirit of the disclosure or equivalent to the subject matter found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

What is claimed is:

1. A catheter for insertion into a patient's vasculature, the catheter extending along a longitudinal axis and comprising:
a catheter body comprising a distal end and a proximal end;
a pump assembly disposed at the distal end of the catheter body and comprising a distal end portion;
a cannula connected to the distal end portion of the pump assembly and comprising a proximal cannula portion and a distal cannula portion;
one or more inlet ports disposed proximal the proximal cannula portion;
one or more outlet ports disposed proximal the distal cannula portion; and
a cage connected to at least one of the cannula and the pump assembly, disposed over the one or more inlet ports, and comprising one or more cage inlet ports, the cage (i) extending along the longitudinal axis and in a circumferential direction about the longitudinal axis and (ii) having an asymmetric geometry, relative to the longitudinal axis, in the circumferential direction that is configured to cause a preferential fluid flow to at least one of the one or more cage inlet ports.

2. The catheter of claim 1, wherein the preferential fluid flow is configured to capture a biomaterial at the at least one of the one or more cage inlet ports.

3. The catheter of claim 2, wherein the at least one of the one or more cage inlets is disposed at a first location along a circumference of a proximal end of the cage, and the remaining locations along the circumference of the proximal end of cage are configured to cause a fluid flow, different from the preferential fluid flow, to the remaining locations.

4. The catheter of claim 3, wherein the preferential fluid flow comprises a higher pressure drop from the at least one of the one or more cage inlet ports to its closest inlet port than a pressure drop from any other remaining location along the circumference of the proximal end of the cage to its respective closest inlet port.

5. The catheter of claim 1, wherein the one or more cage inlet ports are disposed closer to the pump assembly than the one or more inlet ports along the longitudinal axis.

6. The catheter of claim 1, wherein each cage inlet port comprises a distal end spaced at least a predetermined distance from the one or more inlet ports along the longitudinal axis.

7. The catheter of claim 1, wherein at least a portion of the cage is spaced from each inlet port so as to define a conduit that fluidically connects the one or more cage inlet ports with the one or more inlet ports.

8. The catheter of claim 1, wherein the one or more inlet ports are formed in an inlet cage.

9. The catheter of claim 1, wherein the one of the one or more cage inlet ports comprising a first cage inlet port, and wherein the one or more cage inlet ports further comprise one or more second cage inlet ports disposed circumferentially about the cage relative to the first cage inlet port.

10. The catheter of claim 1, wherein the one of the one or more cage inlet ports comprises solely a first cage inlet port.

11. The catheter of claim 9, further comprising a sensor approximately aligned with a distal end of the first cage inlet port along the longitudinal axis.

12. The catheter of claim 1, wherein the pump assembly is configured to pull a fluid through the one or more inlet ports, through a lumen of the cannula, and expel the fluid through the one or more outlet ports.

13. The catheter of claim 1, wherein the pump assembly comprises:
a rotor connected to the distal end of the catheter body; and
one or more impeller blades connected to the rotor.

14. A medical system comprising:
a catheter extending along a longitudinal axis and comprising:
a catheter body comprising a distal end and a proximal end;
a pump assembly disposed at the distal end of the catheter body and comprising a distal end portion;
a cannula connected to the distal end portion of the pump assembly and comprising one or more inlet ports and one or more outlet ports; and
a cage connected to at least one of the cannula and the pump assembly so as to surround the one or more inlet ports and comprising one or more cage inlet ports, the cage having an asymmetric geometry, relative to the longitudinal axis, in a circumferential direction that is configured to cause a preferential fluid flow to at least one of the one or more cage inlet ports; and
a controller connected to the proximal end of the catheter body and configured to operate the pump assembly.

15. The medical system of claim 14, wherein the controller comprises a fluid reservoir that is configured to contain a purge fluid, the controller being configured to deliver the purge fluid through the pump assembly.

16. The medical system of claim 15, wherein the controller comprises a fluid supply control for controlling supply of the purge fluid to the pump assembly and a flow rate control for controlling operation of the pump assembly.

17. The medical system of claim 14, wherein the controller is configured to operate the pump assembly to pull a fluid through the one or more inlet ports, through a lumen of the cannula, and expel the fluid through the outlet port.

18. A method of performing a medical procedure comprising:
navigating a catheter to a target location in a heart of a patient, the catheter extending along a longitudinal axis and comprising (i) a catheter body comprising a distal end and a proximal end, (ii) a pump assembly disposed at the distal end of the catheter body and comprising a distal end portion, (iii) a cannula connected to the distal end portion of the pump assembly and comprising one or more inlet ports, one or more outlet ports, and a lumen connecting the one or more inlet ports and the one or more outlet ports, and (iv) a cage connected to at least one of the cannula and the pump assembly so as to surround the one or more inlet ports and comprising a first cage inlet port;
operating the pump assembly to pull blood through the first cage inlet port, through the one or more inlet ports, through the lumen of the cannula, and expel the blood through the one or more outlet ports;
capturing a biomaterial at the first cage inlet port.

19. The method of claim 18, further comprising:
detecting the capture of the biomaterial.

20. The method of claim 18, wherein the cage comprises an asymmetric geometry, relative to the longitudinal axis, in a circumferential direction of the cage.
